SEQUENCE LISTING (22 sequences)

SEQ ID NO: 1 - DNA, 4212 bp, Pyrocystis lunula
ctcaagtaagctgccacgcaagtgtgccatcacctgtgtctcctatagcagtgccccgcaggaccggccatgtctacgcagttcttaacctttttaaccaacgatgcgaagctcgaccccaatattgtcacatacatgaccagggatttgaagctcgaaagcattgccgactttgcccacttgtggacgaccggtgagtatgagaagggcgtccaagatgatattgtccggaaagtgccggccttccaagacacatcgaaggccccatccaaggtgcagattgcacggcttcgtgctgcatggctatcagcccagcaaagcgaaggggtgccgggagccacggccaaagcagttccagttgcagccaagcctgtcgagcaaaatggcatttgcgaaaagacgggcctggagctgagcggcacggcaaagggtggggcactgagtgcggcacacgtggaacaccttgattctgaagccttccgtgaaggccttcatcagcccaagttccacactgatggcctgcacatgccgcacacttctggcgaaaaaacctacgaaacaggcttccattacctgctcgaggtgcacgaccttggtggcaaaaacaaggatggcggctttggtggcccactttgctccgagccattcagccaagagattgcggacctcgctgaggtcttgctccaggaggcccagaacgacaaaacgctggccttcaccaattttagggatcccgctcccaccctcaccaagaaacaggtggaattgtgcaaggggtttgattacggtgacaaaacgcttaaactgccttgcggcgcgctcccttggccagccggcttgcctgagcctggctatgtgcctcagacgaatccattgcatgggcgttggatcacggtgtccggcggccaggctgccttcatcaagaaggccatcgaggaagggatgctcggcgccgctgaggcgggaaagatcatggccgacacagaccaccaccagactggcggcatgttcctgcgcatcaaccaatttaatgaggtttgcaccgtcgatgcctccgtagctaaatttgcacgtgccaaacgcacatggaagtctggccactacttctatgagccacttgtctccggcggcaacctttttggcgtctgggtgttgcctgaagagtaccgcaagattggttttttttgggagatggagtcgggcaggtgtttccgcatcgaacggcgggctttcgagcgtgatggcctcatgatcatgcggcagtccactgagatcggtggtaacgtctcttttgtcttttacgtcaaggtgtccaacgacccggagtcccagaccatcccactgcagagccgtgactacactgccttggcaggccatgacaatgctcctgacaatttgggcaagccctacccgtgcactgccaaagatctcgactttccgctcaagcgagacacctggcttgacaaaaaccaggaagagatgaacaagcagcgcaccatggtgagcgctcactttgccgacgtctgtgacaatggcttcgaagtccatgacaatctcaagggtgggccggtgagcgccgcgcatgttgaggcgcttggcaaggagcattttgaggatggactgcataagccaaagttccatgatgatggattgcacaagcccatggaagctggcggcaaagtgtacgagactggcttccactacctgctggaggcacacgagcttgggggcaaaaacgccgacggaggcttcggtggcccgctctgtgaggacccgtatgaggatgaggtgcaaagcatgacagagaacttacttgcacagtctgagattgacaacacccttgccttccagaacttcaaggccccctgccctgcccttaccacgaagcaggttgcaatgtgcaagggttttgattacggcaacaagaggctgaaactgccctgtgggccgctcccttggcctgccggtctgcctgcacctggctatgtgccaaagactgacccccttcacgggcgttggatcaccgtctcaggcggccagactgcctttatcaaggaggccatcaagtcaggtatgctcggtgcctccgaggccaagaagatcattgcagacaccgaccatcaccagactggtggcatgtacctgcgcatcaaccagcatggggatgtgtgcactgttgacgcgtcagttgccaaatttgctcgcgccaagcgtacctggaagtctggccactacttctatgagcctcttgtctcaggaggaaatctccttggtgtctgggtactgccggaggagtaccgcaagatcggcttcttttgggagatggagtctggaaagtgtttccgcatcgagcgccgcgcatacccagttggcccctacatgttcctgcgccaagccaccgagatcaacggaaaagtctccttcgtcttctacgtcaaagtctccaacgatccagaatccaagcccatccctttgcagagccgagattacacggccttagctggtgtggacaatgcgcctgataacctggggaatccctatcccactaatgcctacgacttggactaccccaaggagcgggacacgtggcttgacgccaacaaggatgccatgcttgatcagcgtgaaaaagtctctgaagccttcgccgatgtttgtgagaagggattcgaagtcggcgacaatgtcaagggcggcccactgaactccaagcagctcgaaaaatatggtgataacttcaaggatggcatgcaccagcccaccttccacgacgagggcttgcacaaaccgatggaggcaggcggcaagaccttcgagagtggcttccactacttgctggagtgccatgagctcggtggcaagaatgccagcggtggctatggtggtccgctgtgcgaggacccctacggcgctgaagtttctaagttggtggaccaggtcctgaaggactcagacaatgaccgtaccctttgttacaataatttccatgatccttgccctgagctcaccaaggggcaggtggcgatgtgtaagggattcgattacggcgacaagactctcaagctgccttgcggaccgctgccttggccggctggttgcccggagcctggttatgtgcccaagaccaatcctctgcacggtcgatggatcactgtttcgggtggtcaggcggcattcatcaaggaagccatcaagagcggaatgctgggcgccgccgaagcaaacaagatcgccgcggataccgaccatgaacagacgggcggtatgttcttgcgcatcaatcaattcggtgaccagtgcactgttgatgcctcggtggccaagtatgcacgagccaagcgcacctggaggtccggccattacttctatgaaccgcttgtctctggtgggaaccttctcggtgtctgggtgcttcctgaagagtatcgcaagatcggctttttctgggagatggagtctggccggtgtttccgtattgagcgccgcgccttcccagtcggcccctacaccttcttgcgccaggcaacagaggtcaatggcacgatctcttttgttctctatgtaaaggtatcgaacgaccctgagtccaagcctatcccagtgcagagccgtgactacacagccttggccggctgtgacaatgtctgcaccaacttgggcaagccttatccatgcactgccaaagacttggattacccaaacaagcgagacacgtggctcgaccagaacgagaaggagatgattcaccagcggggccttgtggccacaagctttaaagcatgaggcactgcgcttttcttgtcagagcactctgacagtgggcagccgcaccttggaaacggagctttccagcgatgcagcggtgccctcgatatgagcaccatggacatgaatgcaacacccatggatgcagccacataactgccccccggccttcctagccaaccgcttagcagaccttgcggcattcaagtggcgcacgaagtggtgccgatggcgtcaacttcatgcatggatgctcagggcatctggaacttcaagcgtgtggatcagtccacatattcatagtcttgtatgagtcgctggaattgtttgtctgaatcaaccagcgacactaattcaaattatgagccacatggtgtggtttagcagttatacgcgcaatcgtgccatcaaccaaaggatcttgcagcctggaagcatcatcatcag

SEQ ID NO: 2 - protein, 1237 aa, Pyrocystis lunula
MSTQFLTFLTNDAKLDPNIVTYMTRDLKLESIADFAHLWTTGEYEKGVQDDIVRKVPAFQDTSKAPSKVQIARLRAAWLSAQQSEGVPGATAKAVPVAAKPVEQNGICEKTGLELSGTAKGGALSAAHVEHLDSEAFREGLHQPKFHTDGLHMPHTSGEKTYETGFHYLLEVHDLGGKNKDGGFGGPLCSEPFSQEIADLAEVLLQEAQNDKTLAFTNFRDPAPTLTKKQVELCKGFDYGDKTLKLPCGALPWPAGLPEPGYVPQTNPLHGRWITVSGGQAAFIKKAIEEGMLGAAEAGKIMADTDHHQTGGMFLRINQFNEVCTVDASVAKFARAKRTWKSGHYFYEPLVSGGNLFGVWVLPEEYRKIGFFWEMESGRCFRIERRAFERDGLMIMRQSTEIGGNVSFVFYVKVSNDPESQTIPLQSRDYTALAGHDNAPDNLGKPYPCTAKDLDFPLKRDTWLDKNQEEMNKQRTMVSAHFADVCDNGFEVHDNLKGGPVSAAHVEALGKEHFEDGLHKPKFHDDGLHKPMEAGGKVYETGFHYLLEAHELGGKNADGGFGGPLCEDPYEDEVQSMTENLLAQSEIDNTLAFQNFKAPCPALTTKQVAMCKGFDYGNKRLKLPCGPLPWPAGLPAPGYVPKTDPLHGRWITVSGGQTAFIKEAIKSGMLGASEAKKIIADTDHHQTGGMYLRINQHGDVCTVDASVAKFARAKRTWKSGHYFYEPLVSGGNLLGVWVLPEEYRKIGFFWEMESGKCFRIERRAYPVGPYMFLRQATEINGKVSFVFYVKVSNDPESKPIPLQSRDYTALAGVDNAPDNLGNPYPTNAYDLDYPKERDTWLDANKDAMLDQREKVSEAFADVCEKGFEVGDNVKGGPLNSKQLEKYGDNFKDGMHQPTFHDEGLHKPMEAGGKTFESGFHYLLECHELGGKNASGGYGGPLCEDPYGAEVSKLVDQVLKDSDNDRTLCYNNFHDPCPELTKGQVAMCKGFDYGDKTLKLPCGPLPWPAGCPEPGYVPKTNPLHGRWITVSGGQAAFIKEAIKSGMLGAAEANKIAADTDHEQTGGMFLRINQFGDQCTVDASVAKYARAKRTWRSGHYFYEPLVSGGNLLGVWVLPEEYRKIGFFWEMESGRCFRIERRAFPVGPYTFLRQATEVNGTISFVLYVKVSNDPESKPIPVQSRDYTALAGCDNVCTNLGKPYPCTAKDLDYPNKRDTWLDQNEKEMIHQRGLVATSFKA

SEQ ID NO: 3 - DNA, 1146 bp, Pyrocystis lunula
  misc_feature (0)...(0): Domain 3
atggccttcgccgatgtttgtgagaagggattcgaagtcggcgacaatgtcaagggtggcccactgaactccaagcagctcgaaaaatatggtgataacttcaaggatggcatgcaccagcccaccttccacgacgagggcttgcacaaaccgatggaggcaggaggcaagaccttcgagagtggcttccactacttgctggagtgccatgagctcggcggcaagaatgccaccggtggctatggtggtccgctgtgcgaggacccctacggcgctgaagtttctaagttggtggaccaggtcctgaaggactcagacagtgaccgtaccctttgttacaataatttccatgatccttgccctgagctcaccaaggggcaggtggcgatgtgtaagggattcgattatggagacaagactctcaagctgccttgcggcccgttgccttggccggctggttgtccggagcctggttatgtgcccaagaccaatcctctgcacggtcgatggatcactgtttcgggtggtcaggctgcattcatcaaggaagccatcaagagcggaatgttgggcgccgccgaagcaaacaagatcgccgcggataccgaccacgaacagacgggcagtatgttcttgcgcatcaatcaattcggtgaccagtgcactgttgatgcctcggtggccaagtatgcacgagccaagcgcacttggaggtccggccattacttctatgaaccgcttgtctctggtgggaaccttctcggtgtctgggtgcttcctgaagagtatcgcaagatcggcttcttctgggagatggagtctggccggtgtttccgtattgagcgccgcgccttcccagtcggcccctacaccttcttgcgccaggcaacagaggtcaatggcacgatctcttttgttctctatgtgaaggtgtcgaacgaccctgagtccaagcctatcccagtgcagagccgtgactacacagccttggccggctgtgacaatgtctgcaccaacttgggcaagccttatccatgcactgccaaagacttggattacccaaacaagcgagacacgtggctcgaccagaacgagaaggagatgattcaccagcggggccttgtggccacaagctttaaagcatga

SEQ ID NO: 4 - protein, 381 aa, Pyrocystis lunula
  DOMAIN (0)...(0): Domain 3
MAFADVCEKGFEVGDNVKGGPLNSKQLEKYGDNFKDGMHQPTFHDEGLHKPMEAGGKTFESGFHYLLECHELGGKNATGGYGGPLCEDPYGAEVSKLVDQVLKDSDSDRTLCYNNFHDPCPELTKGQVAMCKGFDYGDKTLKLPCGPLPWPAGCPEPGYVPKTNPLHGRWITVSGGQAAFIKEAIKSGMLGAAEANKIAADTDHEQTGSMFLRINQFGDQCTVDASVAKYARAKRTWRSGHYFYEPLVSGGNLLGVWVLPEEYRKIGFFWEMESGRCFRIERRAFPVGPYTFLRQATEVNGTISFVLYVKVSNDPESKPIPVQSRDYTALAGCDNVCTNLGKPYPCTAKDLDYPNKRDTWLDQNEKEMIHQRGLVATSFKA

SEQ ID NO: 5 - DNA, 1251 bp, Pyrocystis lunula
  misc_feature (0)...(0): 1.1kbp fragment of Domain 3
atggggggttctcatcatcatcatcatcatggtatggctagcatgactggtggacagcaaatgggtcgggatctgtacgacgatgacgataaggtaccaggatctgcagccttcgccgatgtttgtgagaagggattcgaagtcggcgacaatgtcaagggtggcccactgaactccaagcagctcgaaaaatatggtgataacttcaaggatggcatgcaccagcccaccttccacgacgagggcttgcacaaaccgatggaggcaggaggcaagaccttcgagagtggcttccactacttgctggagtgccatgagctcggcggcaagaatgccaccggtggctatggtggtccgctgtgcgaggacccctacggcgctgaagtttctaagttggtggaccaggtcctgaaggactcagacagtgaccgtaccctttgttacaataatttccatgatccttgccctgagctcaccaaggggcaggtggcgatgtgtaagggattcgattatggagacaagactctcaagctgccttgcggcccgttgccttggccggctggttgtccggagcctggttatgtgcccaagaccaatcctctgcacggtcgatggatcactgtttcgggtggtcaggctgcattcatcaaggaagccatcaagagcggaatgttgggcgccgccgaagcaaacaagatcgccgcggataccgaccacgaacagacgggcagtatgttcttgcgcatcaatcaattcggtgaccagtgcactgttgatgcctcggtggccaagtatgcacgagccaagcgcacttggaggtccggccattacttctatgaaccgcttgtctctggtgggaaccttctcggtgtctgggtgcttcctgaagagtatcgcaagatcggcttcttctgggagatggagtctggccggtgtttccgtattgagcgccgcgccttcccagtcggcccctacaccttcttgcgccaggcaacagaggtcaatggcacgatctcttttgttctctatgtgaaggtgtcgaacgaccctgagtccaagcctatcccagtgcagagccgtgactacacagccttggccggctgtgacaatgtctgcaccaacttgggcaagccttatccatgcactgccaaagacttggattacccaaacaagcgagacacgtggctcgaccagaacgagaaggagatgattcaccagcggggccttgtggccacaagctttaaagcatga

SEQ ID NO: 6 - protein, 416 aa, Pyrocystis lunula
  DOMAIN (0)...(0): 1.1kbp fragment of Domain 3
MGGSHHHHHHGMASMTGGQQMGRDLYDDDDKVPGSAAFADVCEKGFEVGDNVKGGPLNSKQLEKYGDNFKDGMHQPTFHDEGLHKPMEAGGKTFESGFHYLLECHELGGKNATGGYGGPLCEDPYGAEVSKLVDQVLKDSDSDRTLCYNNFHDPCPELTKGQVAMCKGFDYGDKTLKLPCGPLPWPAGCPEPGYVPKTNPLHGRWITVSGGQAAFIKEAIKSGMLGAAEANKIAADTDHEQTGSMFLRINQFGDQCTVDASVAKYARAKRTWRSGHYFYEPLVSGGNLLGVWVLPEEYRKIGFFWEMESGRCFRIERRAFPVGPYTFLRQATEVNGTISFVLYVKVSNDPESKPIPVQSRDYTALAGCDNVCTNLGKPYPCTAKDLDYPNKRDTWLDQNEKEMIHQRGLVATSFKA

SEQ ID NO: 7 - DNA, 1131 bp, Pyrocystis lunula
  misc_feature (0)...(0): Domain 1
tgcgaaaagacgggcctggagctgagcggcacggcaaagggtggggcactgagtgcggcacacgtggaacaccttgattctgaagccttccgtgaaggccttcatcagcccaagttccacactgatggcctgcacatgccgcacacttctggcgaaaaaacctacgaaacaggcttccattacctgctcgaggtgcacgaccttggtggcaaaaacaaggatggcggctttggtggcccactttgctccgagccattcagccaagagattgcggacctcgctgaggtcttgctccaggaggcccagaacgacaaaacgctggccttcaccaattttagggatcccgctcccaccctcaccaagaaacaggtggaattgtgcaaggggtttgattacggtgacaaaacgcttaaactgccttgcggcgcgctcccttggccagccggcttgcctgagcctggctatgtgcctcagacgaatccattgcatgggcgttggatcacggtgtccggcggccaggctgccttcatcaagaaggccatcgaggaagggatgctcggcgccgctgaggcgggaaagatcatggccgacacagaccaccaccagactggcggcatgttcctgcgcatcaaccaatttaatgaggtttgcaccgtcgatgcctccgtagctaaatttgcacgtgccaaacgcacatggaagtctggccactacttctatgagccacttgtctccggcggcaacctttttggcgtctgggtgttgcctgaagagtaccgcaagattggttttttttgggagatggagtcgggcaggtgtttccgcatcgaacggcgggctttcgagcgtgatggcctcatgatcatgcggcagtccactgagatcggtggtaacgtctcttttgtcttttacgtcaaggtgtccaacgacccggagtcccagaccatcccactgcagagccgtgactacactgccttggcaggccatgacaatgctcctgacaatttgggcaagccctacccgtgcactgccaaagatctcgactttccgctcaagcgagacacctggcttgacaaaaaccaggaagagatgaacaagcagcgcaccatggtgagcgctcactttgccgac

SEQ ID NO: 8 - protein, 377 aa, Pyrocystis lunula
  DOMAIN (0)...(0): Domain 1
CEKTGLELSGTAKGGALSAAHVEHLDSEAFREGLHQPKFHTDGLHMPHTSGEKTYETGFHYLLEVHDLGGKNKDGGFGGPLCSEPFSQEIADLAEVLLQEAQNDKTLAFTNFRDPAPTLTKKQVELCKGFDYGDKTLKLPCGALPWPAGLPEPGYVPQTNPLHGRWITVSGGQAAFIKKAIEEGMLGAAEAGKIMADTDHHQTGGMFLRINQFNEVCTVDASVAKFARAKRTWKSGHYFYEPLVSGGNLFGVWVLPEEYRKIGFFWEMESGRCFRIERRAFERDGLMIMRQSTEIGGNVSFVFYVKVSNDPESQTIPLQSRDYTALAGHDNAPDNLGKPYPCTAKDLDFPLKRDTWLDKNQEEMNKQRTMVSAHFAD

SEQ ID NO: 9 - DNA, 1131 bp, Pyrocystis lunula
  misc_feature (0)...(0): Domain 2
gtctgtgacaatggcttcgaagtccatgacaatctcaagggtgggccggtgagcgccgcgcatgttgaggcgcttggcaaggagcattttgaggatggactgcataagccaaagttccatgatgatggattgcacaagcccatggaagctggcggcaaagtgtacgagactggcttccactacctgctggaggcacacgagcttgggggcaaaaacgccgacggaggcttcggtggcccgctctgtgaggacccgtatgaggatgaggtgcaaagcatgacagagaacttacttgcacagtctgagattgacaacacccttgccttccagaacttcaaggccccctgccctgcccttaccacgaagcaggttgcaatgtgcaagggttttgattacggcaacaagaggctgaaactgccctgtgggccgctcccttggcctgccggtctgcctgcacctggctatgtgccaaagactgacccccttcacgggcgttggatcaccgtctcaggcggccagactgcctttatcaaggaggccatcaagtcaggtatgctcggtgcctccgaggccaagaagatcattgcagacaccgaccatcaccagactggtggcatgtacctgcgcatcaaccagcatggggatgtgtgcactgttgacgcgtcagttgccaaatttgctcgcgccaagcgtacctggaagtctggccactacttctatgagcctcttgtctcaggaggaaatctccttggtgtctgggtactgccggaggagtaccgcaagatcggcttcttttgggagatggagtctggaaagtgtttccgcatcgagcgccgcgcatacccagttggcccctacatgttcctgcgccaagccaccgagatcaacggaaaagtctccttcgtcttctacgtcaaagtctccaacgatccagaatccaagcccatccctttgcagagccgagattacacggccttagctggtgtggacaatgcgcctgataacctggggaatccctatcccactaatgcctacgacttggactaccccaaggagcgggacacgtggcttgacgccaacaaggatgccatgcttgatcagcgtgaaaaagtctctgaagccttcgccgat

SEQ ID NO: 10 - protein, 377 aa, Pyrocystis lunula
  DOMAIN (0)...(0): Domain 1
VCDNGFEVHDNLKGGPVSAAHVEALGKEHFEDGLHKPKFHDDGLHKPMEAGGKVYETGFHYLLEAHELGGKNADGGFGGPLCEDPYEDEVQSMTENLLAQSEIDNTLAFQNFKAPCPALTTKQVAMCKGFDYGNKRLKLPCGPLPWPAGLPAPGYVPKTDPLHGRWITVSGGQTAFIKEAIKSGMLGASEAKKIIADTDHHQTGGMYLRINQHGDVCTVDASVAKFARAKRTWKSGHYFYEPLVSGGNLLGVWVLPEEYRKIGFFWEMESGKCFRIERRAYPVGPYMFLRQATEINGKVSFVFYVKVSNDPESKPIPLQSRDYTALAGVDNAPDNLGNPYPTNAYDLDYPKERDTWLDANKDAMLDQREKVSEAFAD

SEQ ID NO: 11 - DNA, 32 bp, Artificial Sequence
  Primer PID3-F-NcoI+Koz
aagccaccatggccttcgccgatgtttgtgag

SEQ ID NO: 12 - DNA, 32 bp, Artificial Sequence
  Primer PID3-R-XbaI
cctctagatcatgctttaaagcttgtggccac

SEQ ID NO: 13 - DNA, 29 bp, Artificial Sequence
  Primer PID3-F-XbaI+Koz
gctctagaccaccatggccttcgccgatg

SEQ ID NO: 14 - DNA, 27 bp, Artificial Sequence
  Primer PID3-R-EcoRI
ggattctcatgctttaaagcttgtggc

SEQ ID NO: 15 - DNA, 29 bp, Artificial Sequence
  Primer PID3-F-NheI+Koz
ctagctagccaccatggccttcgccgatg

SEQ ID NO: 16 - DNA, 32 bp, Artificial Sequence; identical to SEQ ID NO: 12
  Primer PID3-R-XbaI

SEQ ID NO: 17 - DNA, 37 bp, Artificial Sequence
  Primer DL-F-EcoRI+Koz
ggaattctaaaaatgtctgtttgtgagaagggattcg

SEQ ID NO: 18 - DNA, 32 bp, Artificial Sequence
  Primer DL-R-XbaI(deltaHindIII)
gctctagatcatgctttaaaacttgtggccac

SEQ ID NO: 19 - DNA, 1224 bp, Artificial Sequence
  Dinoflagellate luciferase with His tag for expression in Yeast
atgtctgtttgtgagaagggattcgaagtcggcgacaatgtcaagggtggcccactgaactccaagcagctcgaaaaatatggtgataacttcaaggatggcatgcaccagcccaccttccacgacgagggcttgcacaaaccgatggaggcaggaggcaagaccttcgagagtggcttccactacttgctggagtgccatgagctcggcggcaagaatgccaccggtggctatggtggtccgctgtgcgaggacccctacggcgctgaagtttctaagttggtggaccaggtcctgaaggactcagacagtgaccgtaccctttgttacaataatttccatgatccttgccctgagctcaccaaggggcaggtggcgatgtgtaagggattcgattatggagacaagactctcaagctgccttgcggcccgttgccttggccggctggttgtccggagcctggttatgtgcccaagaccaatcctctgcacggtcgatggatcactgtttcgggtggtcaggctgcattcatcaaggaagccatcaagagcggaatgttgggcgccgccgaagcaaacaagatcgccgcggataccgaccacgaacagacgggcagtatgttcttgcgcatcaatcaattcggtgaccagtgcactgttgatgcctcggtggccaagtatgcacgagccaagcgcacttggaggtccggccattacttctatgaaccgcttgtctctggtgggaaccttctcggtgtctgggtgcttcctgaagagtatcgcaagatcggcttcttctgggagatggagtctggccggtgtttccgtattgagcgccgcgccttcccagtcggcccctacaccttcttgcgccaggcaacagaggtcaatggcacgatctcttttgttctctatgtgaaggtgtcgaacgaccctgagtccaagcctatcccagtgcagagccgtgactacacagccttggccggctgtgacaatgtctgcaccaacttgggcaagccttatccatgcactgccaaagacttggattacccaaacaagcgagacacgtggctcgaccagaacgagaaggagatgattcaccagcggggccttgtggccacaagctttaaagcatctagagggcccttcgaaggtaagcctatccctaaccctctcctcggtctcgattctacgcgtaccggtcatcatcaccatcaccattga

SEQ ID NO: 20 - protein, 407 aa, Artificial Sequence
  Dinoflagellate luciferase with His tag for expression in Yeast
MSVCEKGFEVGDNVKGGPLNSKQLEKYGDNFKDGMHQPTFHDEGLHKPMEAGGKTFESGFHYLLECHELGGKNATGGYGGPLCEDPYGAEVSKLVDQVLKDSDSDRTLCYNNFHDPCPELTKGQVAMCKGFDYGDKTLKLPCGPLPWPAGCPEPGYVPKTNPLHGRWITVSGGQAAFIKEAIKSGMLGAAEANKIAADTDHEQTGSMFLRINQFGDQCTVDASVAKYARAKRTWRSGHYFYEPLVSGGNLLGVWVLPEEYRKIGFFWEMESGRCFRIERRAFPVGPYTFLRQATEVNGTISFVLYVKVSNDPESKPIPVQSRDYTALAGCDNVCTNLGKPYPCTAKDLDYPNKRDTWLDQNEKEMIHQRGLVATSFKASRGPFEGKPIPNPLLGLDSTRTGHHHHHH

SEQ ID NO: 21 - DNA, 1383 bp, Artificial Sequence
  Dinoflagellate luciferase with His tag and secretion signal for expression in Yeast
atgaaggttttgattgttttgttagctatttttgctgccttgccattggctgctcaaccagttattaatactacagttggttctgctgccgaaggttctttggataaaagagaagctggatccactagtaacggccgccagtgtgctggaattctaaaaatgtctgtttgtgagaagggattcgaagtcggcgacaatgtcaagggtggcccactgaactccaagcagctcgaaaaatatggtgataacttcaaggatggcatgcaccagcccaccttccacgacgagggcttgcacaaaccgatggaggcaggaggcaagaccttcgagagtggcttccactacttgctggagtgccatgagctcggcggcaagaatgccaccggtggctatggtggtccgctgtgcgaggacccctacggcgctgaagtttctaagttggtggaccaggtcctgaaggactcagacagtgaccgtaccctttgttacaataatttccatgatccttgccctgagctcaccaaggggcaggtggcgatgtgtaagggattcgattatggagacaagactctcaagctgccttgcggcccgttgccttggccggctggttgtccggagcctggttatgtgcccaagaccaatcctctgcacggtcgatggatcactgtttcgggtggtcaggctgcattcatcaaggaagccatcaagagcggaatgttgggcgccgccgaagcaaacaagatcgccgcggataccgaccacgaacagacgggcagtatgttcttgcgcatcaatcaattcggtgaccagtgcactgttgatgcctcggtggccaagtatgcacgagccaagcgcacttggaggtccggccattacttctatgaaccgcttgtctctggtgggaaccttctcggtgtctgggtgcttcctgaagagtatcgcaagatcggcttcttctgggagatggagtctggccggtgtttccgtattgagcgccgcgccttcccagtcggcccctacaccttcttgcgccaggcaacagaggtcaatggcacgatctcttttgttctctatgtgaaggtgtcgaacgaccctgagtccaagcctatcccagtgcagagccgtgactacacagccttggccggctgtgacaatgtctgcaccaacttgggcaagccttatccatgcactgccaaagacttggattacccaaacaagcgagacacgtggctcgaccagaacgagaaggagatgattcaccagcggggccttgtggccacaagctttaaagcatctagagggcccttcgaaggtaagcctatccctaaccctctcctcggtctcgattctacgcgtaccggtcatcatcaccatcaccattga

SEQ ID NO: 22 - protein, 460 aa, Artificial Sequence
  Dinoflagellate luciferase with His tag and secretion signal for expression in Yeast
MKVLIVLLAIFAALPLAAQPVINTTVGSAAEGSLDKREAGSTSNGRQCAGILKMSVCEKGFEVGDNVKGGPLNSKQLEKYGDNFKDGMHQPTFHDEGLHKPMEAGGKTFESGFHYLLECHELGGKNATGGYGGPLCEDPYGAEVSKLVDQVLKDSDSDRTLCYNNFHDPCPELTKGQVAMCKGFDYGDKTLKLPCGPLPWPAGCPEPGYVPKTNPLHGRWITVSGGQAAFIKEAIKSGMLGAAEANKIAADTDHEQTGSMFLRINQFGDQCTVDASVAKYARAKRTWRSGHYFYEPLVSGGNLLGVWVLPEEYRKIGFFWEMESGRCFRIERRAFPVGPYTFLRQATEVNGTISFVLYVKVSNDPESKPIPVQSRDYTALAGCDNVCTNLGKPYPCTAKDLDYPNKRDTWLDQNEKEMIHQRGLVATSFKASRGPFEGKPIPNPLLGLDSTRTGHHHHHH

(19) United States
(12) Patent Application Publication
(10) Pub. No.: US 2006/0024773 A1
(43) Pub. Date: Feb. 2, 2006

(54) Method for measuring intracellular gene transcription using blue luciferase from dinoflagellate

(75) Inventors: Yoshihiro Ohmiya, Ikeda-shi (JP); Yoshihiro Nakajima, Ikeda-shi (JP); Chie Suzuki, Tsukuba-shi (JP); Masayuki Ryufuku, Tsukuba-shi (JP)

Correspondence Address: KNOBBE MARTENS OLSON & BEAR LLP, 2040 MAIN STREET, FOURTEENTH FLOOR, IRVINE, CA, 92614, US

(73) Assignees: NATIONAL INSTITUTE OF ADVANCED INDUSTRIAL SCIENCE, Tokyo-to (JP); TOYO B-Net Co., Ltd., Tokyo-to (JP)

(21) Appl. No.: 10/996,104

(22) Filed: Nov. 23, 2004

(30) Foreign Application Priority Data
Dec. 10, 2003 (JP) 2003-411489

(51) Int. Cl. C12Q 1/66; C07H 21/04; C12P 21/06; C12N 9/06; C12N 1/12; C12N 5/06; C12N 15/74

(52) U.S. Cl. 435/8; 435/69.1; 435/191; 435/257.1; 435/348; 435/320.1; 536/23.2

(57) ABSTRACT

The present invention relates to a gene construct incorporating any of dinoflagellate luciferase genes into mammalian cells to ensure stable expression.

Method for determining intracellular gene transcription activity using blue light-emitting enzyme from luminescent dinoflagellate

### TECHNICAL FIELD

The present invention relates to a gene construct for detecting a gene transcription activity in a eukaryotic cell using a light-emitting enzyme from luminescent dinoflagellate, an expression vector containing the construct, a transformed eukaryotic cell containing the construct or the expression vector, and a system for singly or dually determining the transcription activity of promoters with the use of the eukaryotic cells.

### BACKGROUND ART

In the life science field, a transcription activity of an intracellular gene has been generally determined, and used for evaluation of exogenous factors given to cells, and analyses of intracellular signal transduction or expression of an individual protein group. The gene transcription activity has been directly determined by Western blotting and the like, or indirectly determined using photoprotein genes or light-emitting enzyme gene as a reporter gene. In particular, it has been generalized to quantify the transcription activity based on relative light unit using a firefly luciferase gene. A fluorescent protein exhibits a fluorescent activity without need of a cofactor almost simultaneously with its intracellular expression. The fluorescent proteins have been used as a monitor protein for examining a localization of a protein by the use of the fluorescent activities in the cell as an indicator, but it is difficult to quantify it, and it is unlikely to use it as the reporter gene for the gene expression.

It is important to analyze a quantitative and temporal dynamic change of the protein gene expression, but the gene transcription activity has been primarily analyzed in conventional reporter techniques.

Recently, a system (dual assay system, Promega) for determining two transcription activities by introducing two gene constructs into the cell, i.e., A transcription active region being inserted in a firefly luciferase gene and B transcription active region being inserted in a Renilla luciferase gene, has been commercially available. However, the Renilla luciferin used in this method has a higher background in chemical luminescence than the Firefly luciferin, and it is difficult to accurately determine a slight change in the transcription activity. Therefore, the Renilla luciferase gene is useful as a control expression gene, but is unsuitable for the analysis of the subject gene.

Multiple signals are trafficked in the cell, and it is essential to construct a technique to quantitatively determine the multiple transcription activities.

An optimal pH of the light-emitting enzyme (derived from luminescent beetle, Renilla and marine ostracod) used for the determination of the gene transcription activity in the eukaryote is from neutral to alkali of pH 7-8, there is no light-emitting enzyme having an acidic optimal pH. When determining the gene transcription activity in the eukaryote such as yeast which grows under the acidic pH, the light-emitting enzyme with acidic optimal pH is desirable but no suitable light-emitting enzyme is available.

As a blue light emitting enzyme, a luciferase from dinoflagellate has been cloned and the structure thereof has been determined (JP 2002-335961-A; Morishita H., Ohashi, S., Oku T., Nakajima Y., Kojima S., Ryufuku M., Nakamura H., and Ohmiya Y: Cloning and Characterization of an Active Fragment of Luciferase from a Luminescent Marine Alga, Pyrocystis lunula. Photochem. Photobiol., 75:311-315, 2002 (partial sequence is described); Okamoto O. K., Liu, Liyun, Robertson D. L., and Hastings J. Woodland: Members of a Dinoflagellate Luciferase Gene Family Differ in Synonymous Substitution Rates. Biochemistry 40:15862-15868, 2001 (entire sequence is described, SEQ ID NO:1). Its expression has been identified in Escherichia coli which is a prokaryote, but no expression has been identified in eukaryotic cells including mammalian cells. A dinoflagellate luciferin has the structure based on a tetrapyrrole ring, which is quite different from the basic structure of the bioluminescence system practically applied hitherto. This enzyme is also reported to have a high enzymatic activity under the acidic pH. However, there is no example where this bioluminescence system has successfully determined the gene transcription activity in the eukaryote including the mammalian cells.

### BRIEF DESCRIPTION OF THE DRAWINGS

FIG. 1 shows transcription activity amounts of a dinoflagellate luciferase gene construct, pcDNA3.1-DL introduced into various mammalian cells.

FIG. 2 shows a correlation of an amount of a dinoflagellate luciferase gene plasmid DNA, pcDNA3.1-DL with relative light unit in a mammalian cell line, NIH3T3.

FIG. 3 shows a reaction curve of a dinoflagellate luciferase produced in a mammalian cell line, NIH3T3.

FIG. 4A shows a correlation of different concentrations of a dinoflagellate luciferase in a mammalian cell line, NIH3T3 with relative light unit.

FIG. 4B shows a correlation of different concentrations of a dinoflagellate luciferase in a mammalian cell line, NIH3T3 with relative light unit.

FIG. 5 shows stability of a dinoflagellate luciferase in a living mammalian cell line, NIH3T3.

FIG. 6A shows a serum concentration dependency of self-luminescence of a dinoflagellate luciferin and a Renilla luciferin.

FIG. 6B shows an example where a transcription activity of E54 element in PerI promoter, the transcription activity of ROR element in BmalI promoter and an internal control in a clock gene were determined and standardized by a firefly luciferase, Renilla luciferase and a dinoflagellate luciferase, respectively in a mammalian cell line, NIH3T3.

FIG. 7 shows luciferin concentration dependency of a dinoflagellate luciferase produced in a mammalian cell line, NIH3T3.

FIG. 8 shows pH dependency of a luminescence activity of a dinoflagellate luciferase produced in a mammalian cell line, NIH3T3 and Escherichia coli.

FIG. 9 shows a correlation of a dinoflagellate luciferase gene construct in which a different transcription factor has been inserted in a mammalian cell line, NIH3T3 with relative light unit.

FIG. 10 shows an example where a transcription activity of a clock gene Per was determined and standardized by an internal control of TK promoter (a reporter gene+a control gene) A: firefly luciferase+Renilla luciferase gene, B: firefly luciferase+dinoflagellate luciferase gene as a control gene, C: dinoflagellate luciferase gene+Renilla luciferase gene.

FIG. 11 shows an example where the expression of a firefly luciferase gene and a dinoflagellate luciferase gene produced in a mammalian cell line, NIH3T3 was simultaneously determined by mixing two luciferin thereof. A: 20 μl of dinoflagellate luciferase lysate and 0.35 μl of firefly luciferase lysate, B: 15 μl of dinoflagellate luciferase lysate and 1 μl of firefly luciferase lysate.

FIG. 12 shows an example where luminescence spectra of a dinoflagellate luciferase and a firefly luciferase co-expressed in a mammalian cell line, NIH3T3 were determined and standardized. A: Luminescence spectra of the dinoflagellate luciferase and the firefly luciferase whose plasmid DNA amount ratios were different. B: Correlation of an expression vector amount of the firefly luciferase with a luminescence intensity at a maximum luminescence wavelength of the standardized firefly luciferase.

FIG. 13 shows an activity of a dinoflagellate luciferase at each collected time in yeast.

### DISCLOSURE OF THE INVENTION

It is an object of the present invention to construct and optimize a dinoflagellate luciferase reporter gene as a blue light emitting enzyme with low background capable of simultaneously determining and quantifying gene transcription activities in eukaryotic cells, alone or by combining other light-emitting enzyme, and further develop a system for determining the gene transcription activity capable of stably determining the reporter enzyme activity to utilize for cell function analyses, treatments/examination of pathology, and new drug development.

In the present invention, a gene construct capable of expressing the light-emitting enzyme from the luminescent dinoflagellate in the eukaryote is made, and a reaction condition where an enzymatic activity thereof alone can be determined or the activity can be determined simultaneously with other light-emitting enzyme activity is optimized.

The present invention provides the gene construct using the following light-emitting enzyme from the luminescent dinoflagellate, mammalian cells, a method for screening drugs using the mammalian cells and the system for determining the transcription activity of each promoter and an optimal reaction determining condition.

1. A gene construct incorporating one or more DNA encoding any light-emitting enzyme of the following (1) to (5) stably expressibly in eukaryotic cells:

(1) a gene encoding a light-emitting enzyme from luminescent dinoflagellate represented by SEQ ID NO:1;

(2) a gene encoding any of active domains 1 to 3 of the light-emitting enzyme from the luminescent dinoflagellate represented by SEQ ID NOS:3, 7 and 9;

(3) a complementary chain to any gene of the above (1) to (2);

(4) a gene capable of hybridizing with any gene of the above (1) to (2) under a stringent condition; and

(5) a gene encoding a polypeptide having substitution, addition, deletion or insertion of one or more amino acid residues in amino acid sequences of SEQ ID NOS:2, 4, 8 and 10, and having a light-emitting enzyme activity.

2. The gene construct according to the above 1 comprising at least one element selected from the group consisting of an element for promoting efficiency of translation and an element for stabilization of mRNA.

3. The gene construct according to the above 1 or 2 further incorporating at least one light-emitting enzyme gene whose luminescence wavelength and luminescence substrate are different from those of the above light-emitting enzyme under the control of a distinct promoter, wherein each luminescence can be distinctively determined.

4. The gene construct according to the above 3 wherein two or more light-emitting enzyme genes incorporated under the control of the distinct promoters comprise one or more genes encoding light-emitting enzyme derived from luminescent dinoflagellate and one or more genes encoding light-emitting enzyme derived from luminescent beetle.

5. The gene construct according to any of the above 1 to 4 incorporating the light-emitting enzymes to be capable of secreting.

6. An expression vector comprising the gene construct according to any of the above 1 to 5.

7. A eukaryotic cell transformed with the gene construct according to any of the above 1 to 5 or the expression vector according to the above 6.

8. A luminescence determining reagent comprising dinoflagellate luciferin and adjusted to pH 5 to 7.5 for determining an enzyme activity of an expressed light-emitting enzymes from luminescent dinoflagellate.

9. The luminescence determining reagent according to the above 8 having a buffer with pH 5 to 7.5.

10. The luminescence determining reagent according to the above 8 wherein a concentration of the dinoflagellate luciferin is adjusted to 10 to 30 μM.

11. A luminescence determining reagent wherein dinoflagellate luciferin and Firefly luciferin coexist in order to simultaneously determine one or more expressed light-emitting enzymes from luminescent dinoflagellate and one or more expressed luciferases from luminescent beetles.

12. A method for determining of a gene transcription activity of a promoter linked to a light-emitting enzyme gene, characterized in that the eukaryotic cells according to the above 7 are cultured, and a luminescence activity in the cultured eukaryotic cells or a disruption solution thereof is determined in the presence of the luminescence determining reagent according to any of the above 8 to 11.

In the present invention, the eukaryotic cells encompass cells widely except for bacteria and cyano bacteria, and include cells of eukaryotes such as yeast, plants and animals (mammals, birds, insects, etc.). Preferable eukaryotes include mammals such as human, cattle, horse, sheep, monkey, swine, mouse, rat, hamster, guinea pig, rabbit and dog, as well as yeast, and preferably human.

For the dinoflagellate luciferase, an entire gene sequence thereof and an entire amino acids thereof are described in Non-patent Reference 2. The dinoflagellate luciferase emits light in the entire amino acid sequence thereof, but has a character as the light-emitting enzyme in any one of domains 1 to 3. Preferable is the gene in the domain 3 described in JP-2002-335961-A.

The genes encoding the domains 1 to 3 have about 90% or more homology one another. The dinoflagellate from which the light-emitting enzyme is derived includes Lingulodinium polyedrnm of Gonyaulaceae family and Pyrocystis lunula of Pyrocytanceae family, and either the light-emitting enzyme derived therefrom or the luminescent domain thereof may be used.

Concretely, the following (1) to (5) of the genes can be used as the dinoflagellate luciferase gene:

(1) a gene encoding the light-emitting enzyme from the luminescent dinoflagellate represented by SEQ ID NO:1;

(2) a gene encoding any of the active domains 1 to 3 of the light-emitting enzyme from the luminescent dinoflagellate represented by SEQ ID NOS:3, 7 and 9;

(3) a complementary chain of the gene of either the above (1) or (2);

(4) a gene capable of hybridizing with the gene of either the above (1) or (2) under a stringent condition; and

(5) a gene encoding a polypeptide having substitution, addition, deletion or insertion of one or more amino acid residues in an amino acid sequence of SEQ ID NO:2, 4, 8 or 10 and having a light-emitting enzyme activity.

As used herein, the “stringent condition” refers to a condition where a specific hybridization is formed whereas no non-specific hybridization is formed. Such a condition is usually about “1×SSC, 0.1% SDS and 37° C.”, preferably about “0.5×SSC, 0.1% SDS and 42° C.”, and more preferably about “0.2×SSC, 0.1% SDS and 65° C.”. DNA obtained by the hybridization has usually high homology with DNA represented by a base sequence described in SEQ ID NO:1, 3, 7 or 9. The high homology indicates 65% or more homology, preferably 75% or more homology, more preferably 90% or more homology and particularly 95% or more homology.

A number of the amino acid residues substituted, added, deleted or inserted is not limited as long as a light-emitting enzyme action is not lost, and is preferably 20 or less, more preferably 15 or less, still more preferably 10 or less, and most preferably 5 or less of amino acid residues.

In one preferable embodiment, the present inventor has found by examining various expression systems that it is useful to introduce at least one element selected from the group consisting an element for promoting efficiency of translation and an element for stabilization of mRNA into the gene construct for stably expressing the light-emitting enzyme in the eukaryotic cells (e.g., mammalian cells, yeast). As the element for promoting the efficiency of the translation, a kozak sequence (Ko) and the like are exemplified, and as the element for the stabilization of mRNA, β-globin intron II and the like are exemplified.

As an expression promoter in the eukaryotic cells (e.g., mammalian cells, yeast), an SV40, CMV, CAG, GAL1 and the like are exemplified, and preferably the CAG by which an expressed amount of the light-emitting enzyme is increased is included.

In one preferable embodiment, the gene construct of the present invention incorporates the light-emitting enzyme from the luminescent dinoflagellate to be capable of secreting. When the gene construct incorporates genes encoding Renilla luciferase, firefly luciferases and luminescent beetle luciferases are incorporated together with the light-emitting enzyme gene from the luminescent dinoflagellate, it is preferable to incorporate these luciferases or the light-emitting enzymes to be capable of secreting. It is preferable to secret these luciferases or light-emitting enzymes because amounts of these enzymes can be quantified without disrupting the eukaryotic cells. To secret the luciferase or the light-emitting enzyme, a leader sequence for the secretion could be ligated before the enzyme sequence, and includes a secretion signal of Renilla luciferase in the mammalian cells (Nakajima Y., Kobayashi K., Yamagishi K., Enomoto T and Ohmiya Y: cDNA cloning and characterization of secreted luciferase from the luminous Japanese ostracod, Crpridina noctiluca. Biosci. Biotechnol. Biochem., 68:565-70, 2004) and a yeast secretion signal in the yeast (Clements M. J., Gatlin G. H., Price M. J., Edwards R. M.: Secretion of human epidermal growth factor Saccharomyces cerevisiae using synthetic leader sequences. Gene, 106:267-272, 1991).

When the dinoflagellate luciferase is expressed in the eukaryotic cells, it has the following characteristics:

(i) it is a reporter gene having a quantitative property where a luminescence activity is increased in correlation with an amount of the introduced gene (expression plasmid) in the eukaryotic cells;

(ii) the luminescence activity is proportional to a concentration of the dinoflagellate luciferase in the presence of a constant amount of the dinoflagellate luciferin;

(iii) the luminescence activity is about 2 times higher at pH 5.0 to 7.5 than that of the luciferase expressed in Escherichia coli; and

(iv) a background is extremely low compared to conventional light-emitting enzymes such as Renilla luciferase.

The dinoflagellate luciferase has the high luminescence activity at a region of pH 5 to 7.5 (weak acidic to weak alkali) in the cells and the extremely low background, and thus is useful as the reporter gene for determining two or more different gene transcription activities.

As the light-emitting enzyme capable of being expressed in the eukaryotic cells in addition to the light-emitting enzyme from the dinoflagellate, Renilla luciferase, firefly luciferases, luminescent beetle luciferases and the like are exemplified.

As used herein, the “light-emitting enzyme” means a photoprotein group such as luciferase which catalyzes a luciferin photochemical reaction, and the light-emitting enzyme includes the photoprotein such as aequorin.

The transformed eukaryotic cells containing the light-emitting enzyme from the dinoflagellate are cultured in an appropriate medium, subsequently disrupted by sonication, and then the luciferin such as dinoflagellate luciferin is added to determine the luminescence activity. When other light-emitting enzyme genes such as Renilla luciferase gene and firefly luciferase gene are expressed simultaneously with the light-emitting enzyme gene from the dinoflagellate as the light-emitting enzymes, it is possible to add corresponding luciferins such as Renilla luciferin and Firefly luciferin.

A concentration of the luciferin such as dinoflagellate luciferin is usually about 1 to 40 μM, and preferably about 10 to 30 μM.

It is preferable to determine a sample such as cell lysate containing the dinoflagellate luciferase preferably at pH 5.0 to 7.5, and more preferably at pH 5.5 to 6.5 because the luminescence activity is high.

### BEST MODE FOR CARRYING OUT THE INVENTION

The present invention will be illustrated in detail below in reference to Examples.

### EXAMPLE 1

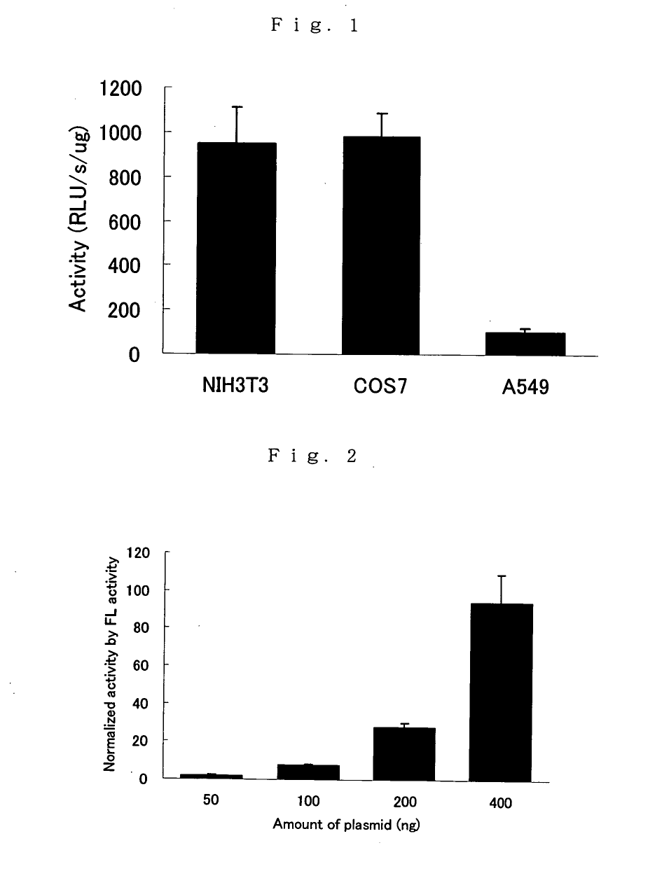

The luciferase genes have been cloned from two luminescent dinoflagellate species, Lingulodinium polyedrnm and Pyrocystis lunula. The luminescent dinoflagellate luciferase is a protein (SEQ ID NO:1) with molecular weight of about 140,000 having three domains containing an enzymatically active site. In these, a 1.1 kbp fragment (SEQ ID NO:5) encoding the dinoflagellate luciferase gene domain 3 was cut out at BglII and EcoRI sites from pTH-P1L (Morishita H., Ohashi, S., Oku T., Nakajima Y., Kojima S., Ryufuku M., Nakamura H., and Ohmiya Y: Cloning and Characterization of an Active Fragment of Luciferase from a Luminescent Marine Alga, Pyrocystis lunula. Photochem. Photobiol., 75:311-315, 2002) containing the domain 3 whose expression had been confirmed in Escherichia coli, and inserted into the BamHI/EcoRI sites of pcDNA3.1/HisC (Invitrogen) with CMV promoter to make pcDNA3.1-DL. NIH3T3 cells, COS7 cells and A549 cells were cultured in DMEM containing 10% FBS at 37° C. in 5% CO₂. The cells were seeded at 6×10⁴ cells/ well in a 24-well plate, cultured for one day, and subsequently, transfected with 200 ng/well of pcDNA3.1-DL using Lipofectamine Plus reagent (Invitrogen). After the transfection, the cells were cultured for two days, then washed once with 500 μL of PBS, and disrupted ultrasonically in 300 μL of 0.1 M phosphate buffer (pH 6.0) to collect lysate. Subsequently, 10 μL of 1 μM dinoflagellate luciferin in 0.1 M phosphate buffer (pH 6.0) was added to 50 μL of the collected lysate, and the luminescence activity for 20 sec was determined by LB9506 (Berthold). At the same time, an protein amount in each lysate was determined using a BCA protein quantification reagent (Pearce), and relative light unit per protein amount was calculated. The high luminescence activity was identified in the NIH3T3 cells and the COS7 cells (FIG. 1). The NIH3T3 cells were seeded at 6×10⁴ cells/well in the 24-well plate, and cultured for one day. The cells were co-transfected with 50, 100, 200 or 400 ng/well of pcDNA3.1-DL together with 10 ng/well of pGV-C2 (Toyo Ink) as an internal standard using the Lipofectamine Plus reagent (Invitrogen). After the transfection, the cells were cultured for one day, then washed once with 500 μL of PBS, and disrupted ultrasonically in 300 μL of 0.1 M phosphate buffer (pH 6.0) to collect lysate. Subsequently, 10 μL of 1 μM dinoflagellate luciferin in 0.1 M phosphate buffer (pH 6.0) was added to 50 μL of the collected lysate, and the luminescence activity for 20 sec was determined by LB9506 (Berthold). Luminescence values obtained of the dinoflagellate luciferase was standardized using the luminescence values of a co-expressed firefly luciferase as a control. The luminescence activity was increased in correlation with the amount of pcDNA3.1-DL used (FIG. 2). This result indicates that the dinoflagellate luciferase is the reporter gene with quantitative property where the relative light unit varies depending on the amount of introduced gene.

### EXAMPLE 2

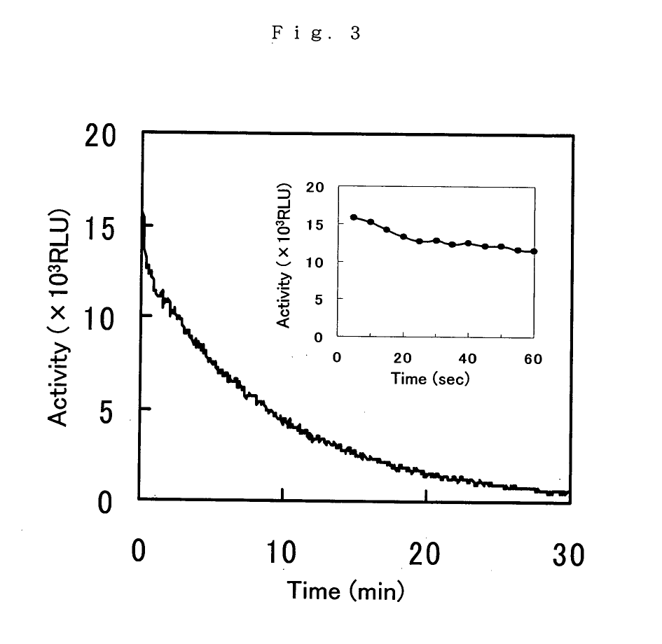

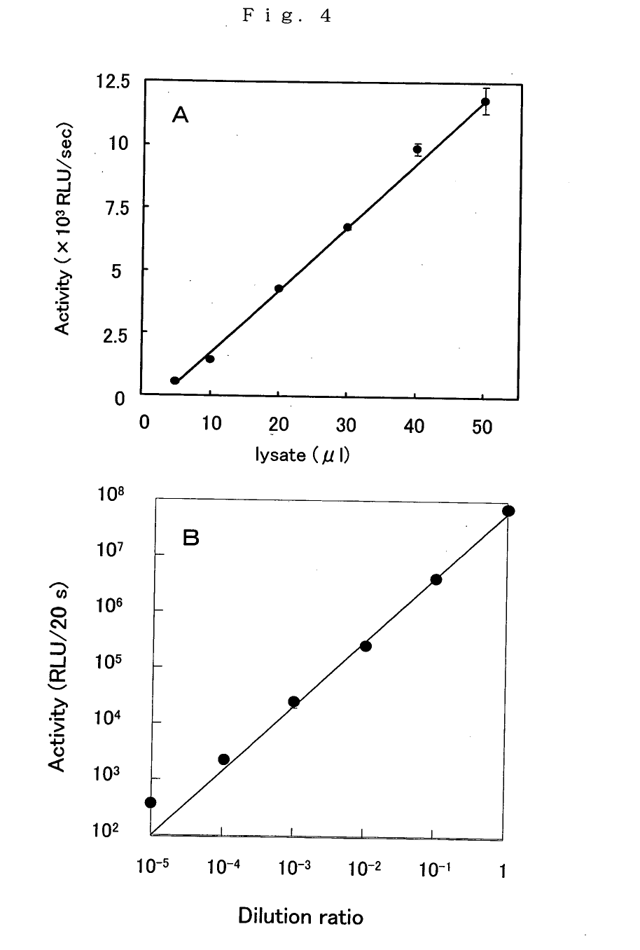

The NIH3T3 cells were seeded at 3×10⁴ cells/well in a 48-well plate. After culturing for one day, the cells were transfected with 250 ng/well of pcDNA3.1-DL using Lipofectamine Plus reagent (Invitrogen). After the transfection, the cells were cultured for two days, then washed once with 150 μL of PBS, and disrupted ultrasonically in 150 μL of 0.1 M phosphate buffer (pH 6.0) to collect lysate. Subsequently, 50 μL of 1 μM dinoflagellate luciferin was added to 50 μL of the collected lysate, and the luminescence activity for 30 min was determined using a luminescencer AB2100 supplied from ATTO Corporation (FIG. 3).

Stable luminescence was observed for about one min after the start of determination, but the activity was progressively reduced and reduced by half after 5 min. The lysate was diluted to be 50, 40, 30, 20, 10, and 5 μL per 50 μL with 0.1M phosphate buffer (pH 6.0). To 50 μL of each diluted lysate, 10 μL of 1 μM dinoflagellate luciferin in 0.1 M phosphate buffer (pH 6.0) was added, the luminescence activity for 20 sec was determined by LB9506 (Berthold), and the activity was plotted to a concentration of the dinoflagellate luciferase (FIG. 4A). The luminescence activity was changed linearly depending on the concentrations of the dinoflagellate luciferase, and revealed a quantitative property as the light-emitting enzyme. Furthermore, the lysate was diluted with 0.1 M phosphate buffer (pH 6.0) to be 10⁻¹, 10⁻², 10⁻³, 10⁻⁴ and 10⁻⁵ times. To 50 μL of each diluted lysate, 10 μL of 2 μM dinoflagellate luciferin in 0.1 M phosphate buffer (pH 6.0) was added, the luminescence activity for 20.sec was determined by LB9506 (Berthold), and the activity was plotted to a concentration of the dinoflagellate luciferase. The lysate exhibited the larger luminescence activity, and the quantitative property in a wide range was shown (FIG. 4B).

### EXAMPLE 3

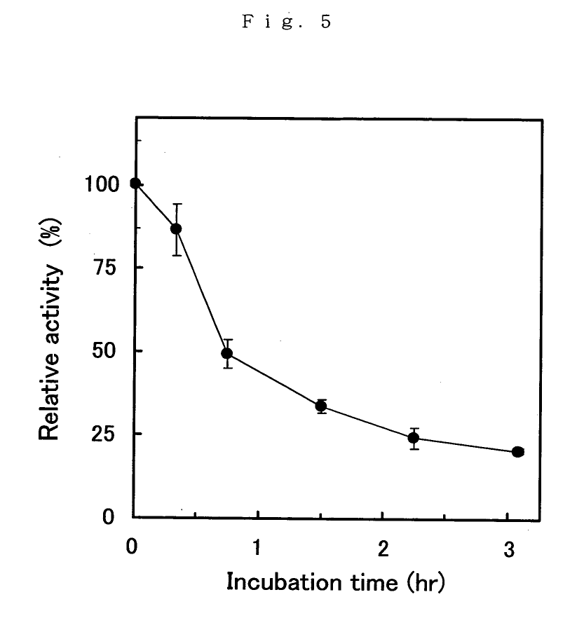

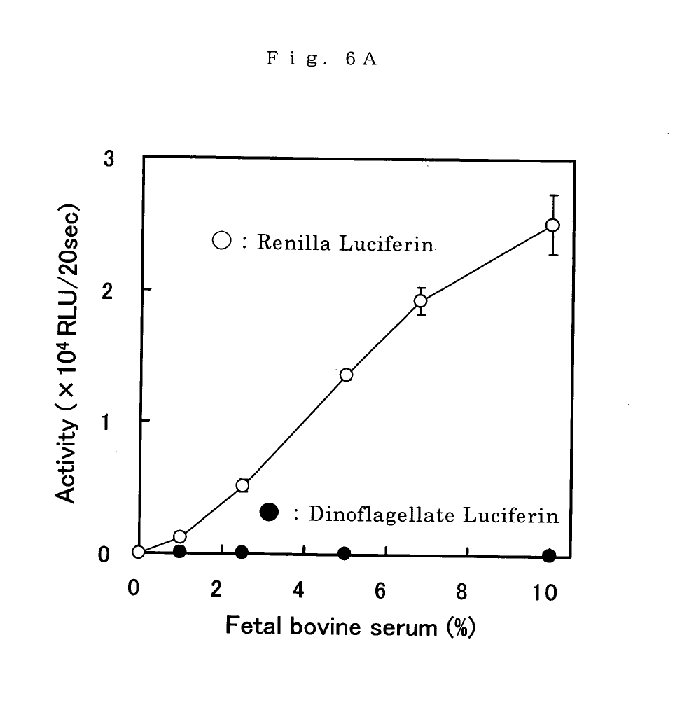

The NIH3T3 cells were seeded at 6×10⁴ cells/well in the 24-well plate. After culturing for one day, the cells were transfected with 200 ng/well of pcDNA3.1-DL using Lipofectamine Plus reagent (Invitrogen). After the transfection, the cells were cultured for one day, and cycloheximide (Sigma) at a final concentration of 250 μM was added to the medium. Thirty minutes after the addition of cycloheximide, the cells were washed with 300 μL of PBS, and making a washed time as 0 hour, the cells were sequentially disrupted ultrasonically in 300 μL of 20 mM Tris buffer (pH 7.5) to collect as the lysate. To 50 μL of each collected lysate, 10 μL of 2 μM dinoflagellate luciferin in 20 mM Tris buffer (pH 7.5) was added, and the luminescence activity for 20 sec was determined by LB9506 (Berthold) (FIG. 5). As a result, a half life of the expressed dinoflagellate luciferase in living cells was about 45 min.

### EXAMPLE 4

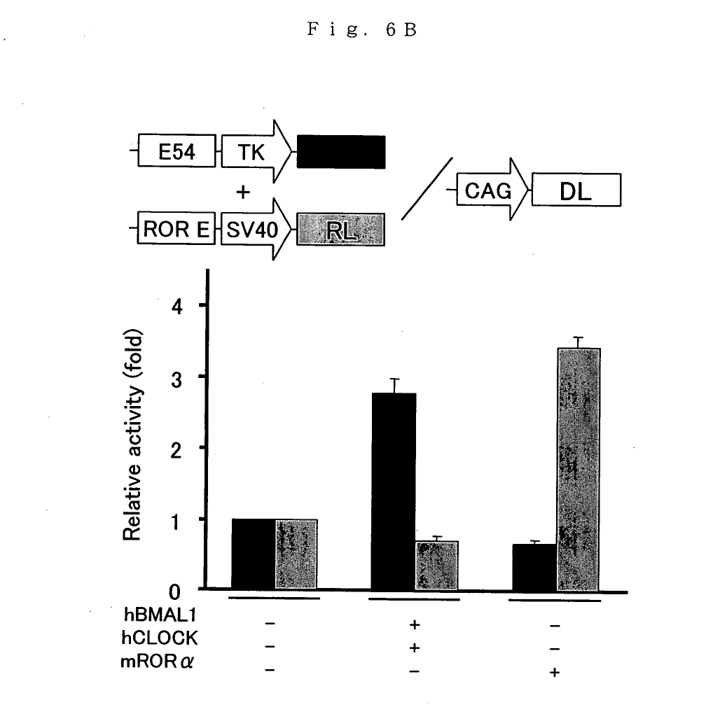

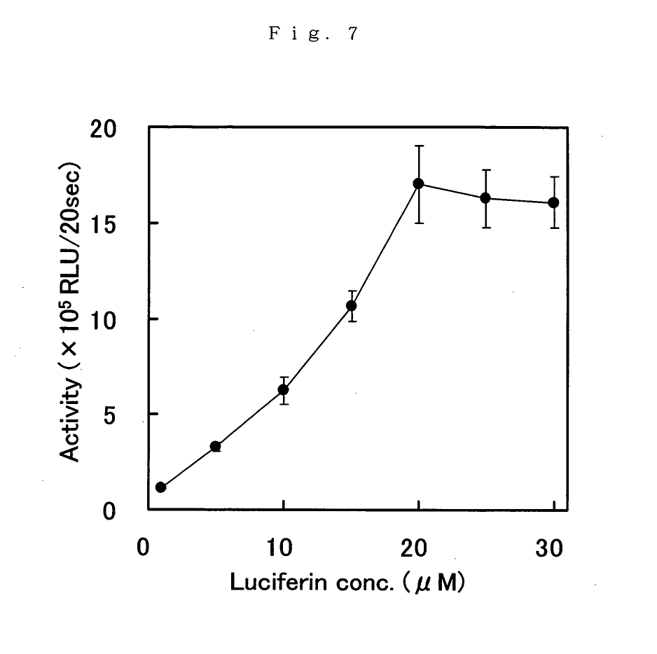

A determining condition of the enzyme activity of the dinoflagellate luciferase expressed in the mammalian cells was examined. To confirm a background of the dinoflagellate luciferin, serum was diluted with 20 mM Tris buffer (pH 7.5) to be 0 to 10%. To 50 μL of each diluted solution, 50 μL of 5 μM Renilla luciferin (coelenterazine) or 50 μL of 1 μM dinoflagellate luciferin in 0.1 M phosphate buffer (pH 6.0) was added, and the luminescence activity for 20 sec was determined by LB9506 (Berthold). In the Renilla luciferin, the luminescence activity was increased depending on the serum concentration whereas in the dinoflagellate luciferin, no luminescence was detected (FIG. 6). When the activity of the dinoflagellate luciferase was determined, the enzyme activity could be determined even in the presence of 1 to 10% serum. It could be confirmed that the background of the dinoflagellate luciferase was lower than that of the Renilla luciferase used as the conventional control.

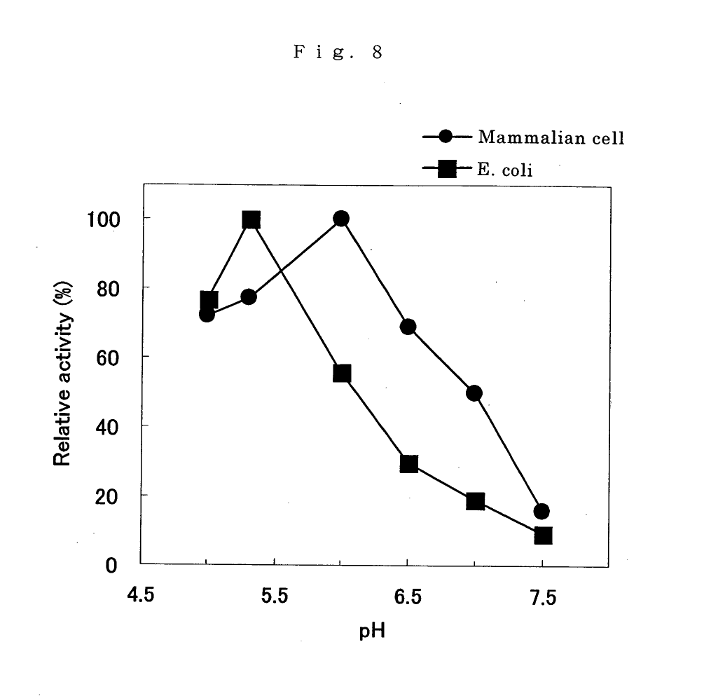

The NIH3T3 cells were seeded at 2.5×10⁵ cells/well in a 35 mm dish. After culturing for one day, the cells were transfected with 1 μg/well of pcDNA3.1-DL using Lipofectamine Plus reagent (Invitrogen). After the transfection, the cells were cultured for two days, then washed once with 1 mL of PBS, and disrupted ultrasonically in 0.1 M phosphate buffer (pH 6.0) to collect lysate. To 50 μL of each collected lysate, 50 μL of dinoflagellate luciferin diluted with 0.1 M phosphate buffer (pH 6.0) at a final concentration of 1, 5, 10, 15, 20, 25, and 30 μM was added, and the luminescence activity for 20 sec was determined by LB9506 (Berthold) (FIG. 7). The high activity was observed at around 20 μM of the luciferin concentration. Therefore, an optimal luciferin concentration is about 10 to 30 μM.

The NIH3T3 cells were seeded at 6×10⁴ cells/well in the 24-well plate. After culturing for one day, the cells were transfected with 400 ng/well of pcDNA3.1-DL using Lipofectamine Plus reagent (Invitrogen). After the transfection, the cells were cultured for two days, then washed once with 500 μL of PBS, and disrupted ultrasonically in 300 μL of purified water to collect lysate. Subsequently, 50 μL of 2 μM dinoflagellate luciferin diluted with 0.1 M phosphate buffer (pH 5.0, 5.5, 6.0, 6.5, 7.0 and 7.5) was added to 50 μL of the collected lysate, and the luminescence activity for 20 sec was determined using LB9506 (Berthold) (FIG. 8). The luminescence activity of the dinoflagellate luciferase expressed in Escherichia coli was also determined under the same condition. As a result, the luciferase produced in Escherichia coli exhibited the maximum relative light unit at pH 5.5 whereas the luciferase produced in the mammalian cells exhibited the maximum at pH 6.0 and kept the enzyme activity of 60% or more at pH 5 to 6.5. This indicates that the luminescence activity can be determined at an acidic side in the dinoflagellate luciferase whereas the optimal pH of the firefly and Renilla luciferases is pH 7 to 8.

### EXAMPLE 5

Concerning the expression of a protein having an activity in the mammalian cells, difference of the luminescence activity due to difference of inserted regions of the promoter which regulates the expression was examined. A DL fragment (SEQ ID NO:3) was amplified by PCR with pcDNA3.1-DL as a template using a primer, PID3-F-NcoI+Koz where a NcoI site, a Koz sequence and an initiation codon had been added at the 5′ end (5′-AAG CCA CCA TGG CCT TCG CCG ATG TTT GTG AG-3′: SEQ ID NO:11) and a primer, PID3-R-XbaI where a XbaI site had been added at the 3′ end (5′-CCT CTA GAT CAT GCT TTA AAG CTT GTG GCC AC-3′: SEQ ID NO:12).

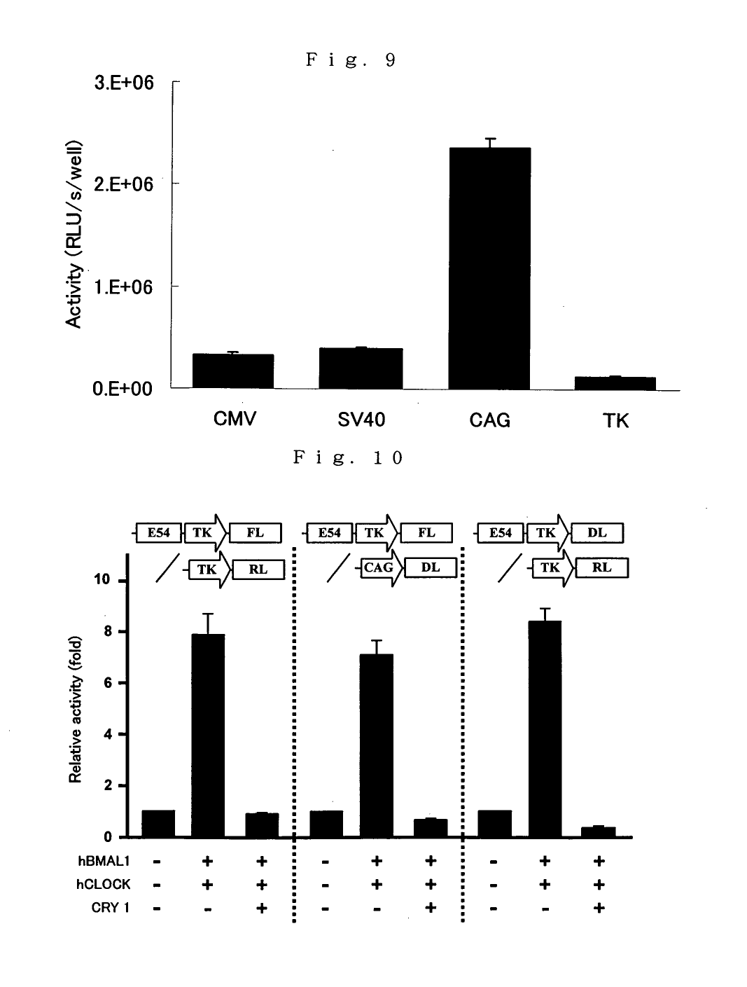

This PCR product was subcloned into pCR2.1-TOPO (Invitrogen), and subsequently the PCR product, DL was inserted at the NcoI/XbaI sites of pGV-C2 (Toyo Ink) from which the firefly luciferase had been cut out at the NcoI/XbaI sites, to make pGC2-DL having an SV40 promoter. The DL fragment was amplified by PCR with pGC2-DL as the template using the primer, PID3-F-XbaI+Koz where the XbaI site and the Koz sequence had been added at the 5′ end (5′-GCT CTA GAC CAC CAT GGC CTT CGC CGA TG-3′: SEQ ID NO:13) and the primer, PID3-R-EcoRI where an EcoRI site had been added at the 3′ end (5′-GGA TTC TCA TGC TTT AAA GCT TGT GGC-3′: SEQ ID NO:14). Meanwhile, a fragment was cut out at BlnI/BspT104 sites from pMK10, a part of IRES and neomycin were removed by self-ligation and the PCR product was inserted thereto by treating with restriction enzymes, XbaI and EcoRI to make pCAG-DL (having a CAG promoter). Furthermore, the DL fragment was amplified by PCR with pGC2-DL as the template using the primer, PID3-F-NheI+Koz where the XbaI site and the Koz sequence had been added at the 5′ end (5′-CTA GCT AGC CAC CAT GGC CTT CGC CGA TG-3′: SEQ ID NO:15) and the primer, PID3-R-XbaI where the XbaI site had been added at the 3′ end (5′-CCT CTA GAT CAT GCT TTA AAG CTT GTG GCC AC-3′: SEQ ID NO:16). This PCR product was subcloned into pCR2.1-TOPO (Invitrogen), and subsequently inserted at the NheI/XbaI sites of phRL-TK (Promega) from which the Renilla luciferase, RL had been removed by cutting out at the NheI/XbaI sites, to make pTK-DL having a TK promoter. The NIH3T3 cells were seeded at 6×10⁴ cells/well in the 24-well plate. After culturing for one day, the cells were transfected with 200 ng/well of pcDNA3.1-DL having the CMV promoter, pGC2-DL having the SV40 promoter, pCAG-DL having the CAG promoter or pTK-DL having the TK promoter using Lipofectamine Plus reagent (Invitrogen). After the transfection, the cells were cultured for two days, then washed once with 500 μL of PBS, and disrupted ultrasonically in 300 μL of sterile water to collect lysate. Subsequently, 50 μL of 20 μM dinoflagellate luciferin (1 M phosphate buffer (pH 6.0)) was added to 50 μL of the collected lysate, the luminescence activity for 20 sec was determined using LB9506 (Berthold), and the amount of emitted light per well was calculated. The luminescence activity could be detected in all cases of the four promoters, and in particular, the high luminescence activity was detected in the case of the CAG promoter (FIG. 9). These results indicate that the dinoflagellate luciferase gene is a reporter gene capable of determining the transcription activity of the different genes.

### EXAMPLE 6A

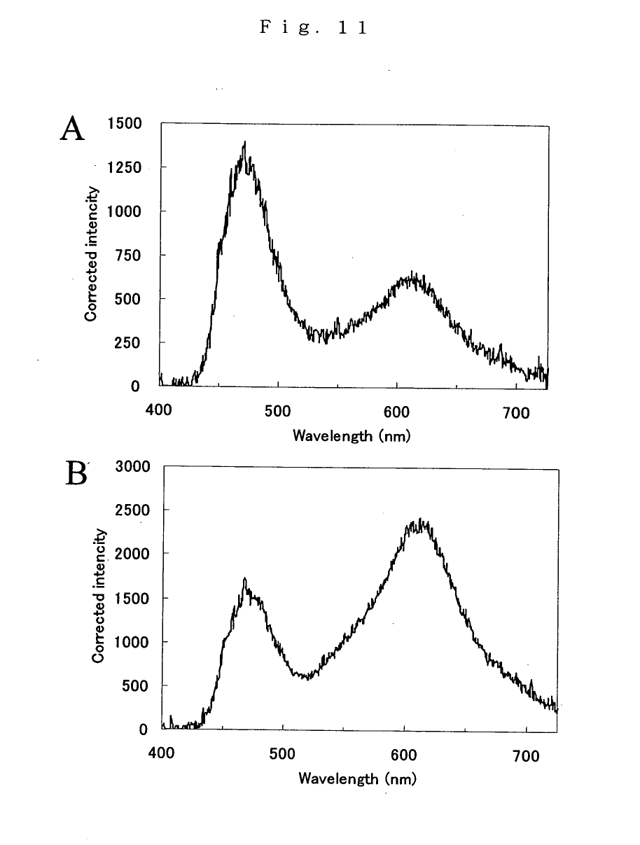

The NIH3T3 cells were seeded at 4.5×10⁴ cells/well in the 48-well plate. After culturing for one day, the cells were co-transfected with 40 ng of a reporter, E54TK-FL (E54 element of Per promoter+firefly luciferase), 40 ng of E54TK-DL (E54 element of Per promoter+dinoflagellate luciferase), 50 ng of a clock gene, hBMAL1, hCLOCK and mCRY1, respectively, and internal controls, 2 ng of phRL-TK (Renilla luciferase) and 20 ng of pCAG-DL using Lipofectamine Plus reagent (Invitrogen). After the transfection, the cells were cultured for one day, then washed once with 200 μL of PBS, and disrupted ultrasonically in 200 μL of DW to collect lysate. To 50 μL of the collected lysate, 50 μL of 20 μM dinoflagellate luciferin (1 M phosphate buffer (pH 6.0) diluted with 0.1 M phosphate buffer for the DL activity, 50 μL of PicaGene luminescence reagent (Toyo Ink) for the firefly luciferase activity or 50 μL of 500 nM coelenterazine diluted with 10 mM Tris buffer (pH 7.4) for the Renilla luciferase activity was added, and the luminescence activity for 20 sec was determined using LB9506 (Berthold) (FIG. 10). A is the result where the firefly luciferase gene is the reporter gene and the Renilla luciferase gene is the control gene, B is the result where the firefly luciferase gene is the reporter gene and the dinoflagellate luciferase gene is the control gene, and C is the result where the dinoflagellate luciferase gene is the reporter gene and the Renilla luciferase gene is the control gene. It is evident that the gene transcription activity can be evaluated in any combinations. These results indicate that the dinoflagellate luciferase can be used as the internal control enzyme of the firefly or Renilla luciferase or that the firefly or Renilla luciferase can be used as the internal control enzyme and the dinoflagellate luciferase can be used as the reporter enzyme.

### EXAMPLE 6B

The NIH3T3 cells were seeded at 2.5×10⁴ cells/well in the 24-well plate. After culturing for one day, the cells were co-transfected with 20 ng of reporter E54TK-FL (E54 element of mouse Perl promoter+firefly luciferase), 20 ng of RORE-RL (REV-ERV/ROR element in mouse Bmal1 promoter+Renilla luciferase), 50 ng of clock gene, human BMAL1, 50 ng of clock gene human CLOCK, 50 ng of clock gene mouse RORα and 20 ng of internal control, pCAG-DL using Lipofectamine Plus reagent (Invitrogen). After the transfection, the cells were cultured for two days, then washed once with 500 μL of PBS, and disrupted ultrasonically in 300 μL of DW to collect lysate. To 50 μL of the collected lysate, 50 μL of 14 μM dinoflagellate luciferin diluted with 0.1 M phosphate buffer (pH 6.0) for the DL activity, 50 μL of PicaGene luminescence reagent (Toyo Ink) for the firefly luciferase activity or 50 μL of 70 nM coelenterazine diluted with 20 mM Tris buffer (pH 7.4) for the Renilla luciferase activity was added, and the luminescence activity for 20 sec was determined using LB9506 (Berthold). And activity values of the firefly and Renilla luciferases which were the reporters were standardized with the activity value of the dinoflagellate luciferase which was the internal control. Until now, it has been known that human BMAL1 and human CLOCK proteins activate the promoter linked to the E54 element and inactivate the promoter linked to the ROR element whereas the mouse RORα inactivates the promoter linked to the E54 element and activates the promoter linked to the ROR element in separated experiments. In the present experiment, the similar difference in transcription activity could be also determined by determining the lysates from the same cells (FIG. 6B). The present results indicate that three or more transcription activities can be determined by combining the dinoflagellate luciferase gene with other two reporter genes which emit the light with different substrates.

### EXAMPLE 7

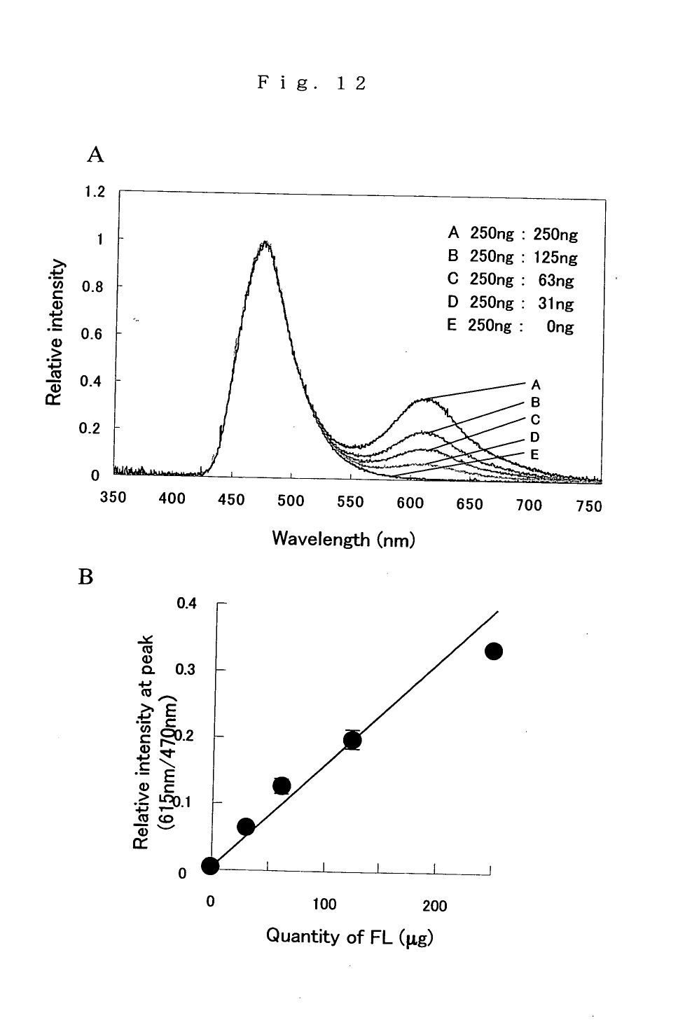

The NIH3T3 cells were seeded at 6×10⁴ cells/well in the 24-well plate. After culturing for one day, the cells were transfected with 400 ng/well of pcDNA3.1-DL or pGV-C2 separately using Lipofectamine Plus reagent (Invitrogen). After the transfection, the cells were cultured for two days, then washed once with 500 μL of PBS, and disrupted ultrasonically in 0.1 M phosphate buffer (pH 6.0) for the dinoflagellate luciferase and in 10 mM Tris buffer (pH 7.5) for the firefly luciferase to collect lysates. Subsequently, 20 μL of the collected lysate for the dinoflagellate luciferase was mixed with 0.35 μL of the lysate for the firefly light-emitting enzyme, and 1 μL of the 10 μM dinoflagellate luciferin and 10 μL of PicaGene luminescence reagent were added thereto to determine luminescence spectrum using a high sensitive spectrometer (ATTO) (FIG. 11A). Also, 15 μL of the lysate for the dinoflagellate luciferase was mixed with 1 μL of the lysate for the firefly luciferase to determine under the same condition (FIG. 11B). The dinoflagellate luciferase has the maximum luminescence wavelength of 470 nm which exhibits a blue color and the firefly luciferase has the maximum luminescence wavelength of 610 nm which exhibits a red color. Therefore, they have the distinct luminescence spectra. The firefly luciferase exhibited a changed color of yellow-green to red because the determination solution is under the acidic pH. Meanwhile, the spectrum is changed depending on the amounts of the added dinoflagellate luciferase and firefly luciferase, and thus an individual enzyme activity amount can be calculated from the luminescence spectra.

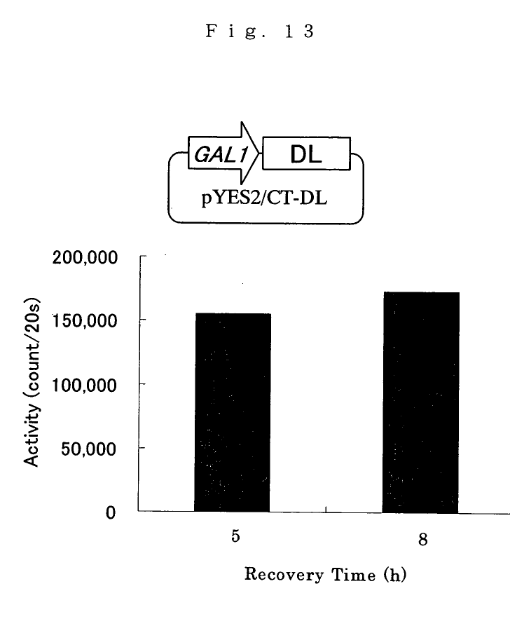

Furthermore, the NIH3T3 cells were seeded at 5×10⁴ cells/well in the 24-well plate. After culturing for one day, the cells were co-transfected with 250 ng of pCAG-DL and 0, 31, 63, 125, or 250 ng of pGV-2 such that a plasmid DNA ratio of DL:FL was 1:1, 2:1, 4:1 or 8:1 using Lipofectamine Plus reagent (Invitrogen). After the transfection, the cells were cultured for two days, then washed once with 500 μL of PBS, and disrupted ultrasonically in DW to collect lysates. Subsequently, 7 μM dinoflagellate luciferin, 470 μM Firefly luciferin, 530 μM ATP, 4 mM MgSO4, 270 μM CoA and 33.3 mM DTT diluted with 0.05 M phosphate buffer (pH 6.0) were added to 15 μL of the collected lysate, the luminescence spectra were determined using the high sensitive spectrometer (ATTO), and standardized using the value of the maximum luminescence wavelength, 470 nm of the dinoflagellate luciferase (FIG. 12A). The dinoflagellate luciferase has the maximum luminescence wavelength of about 470 nm which exhibits a blue color and the firefly luciferase has the maximum luminescence wavelength of about 615 nm which shifts to the long wavelength side under the acidic pH and exhibits a red color. Therefore, they have the distinct luminescence spectra. Relative luminescence intensity (luminescence intensity at 615 nm/luminescence intensity at 470 nm) was plotted to the plasmid DNA amount of the firefly luciferase, and consequently the luminescence activity and the plasmid amount were linearly changed. Thus, it could be confirmed that the expression amount of the firefly luciferase could be standardized and quantified (FIG. 12B). The present results indicate that two light-emitting enzyme activities can be quantitatively and simultaneously determined under the condition where the dinoflagellate luciferin and the Firefly luciferin which are different coexist.

### EXAMPLE 8

The luminescence activity of the dinoflagellate luciferase was confirmed in a yeast expression system. The DL fragment (SEQ ID NO:3) was amplified by PCR with pSV40-DL as a template using a primer, DL-F-EcoRI+Koz, ATG where an EcoRI site, yeast KoZ sequence and an initiation codon had been added at the 5′ end (5′-GGA ATT CTA AAA ATG TCT GTT TGT GAG AAG GGA TTC G-3′: SEQ ID NO:17) and a primer, DL-R-XbaI (ΔHindIII) where a XbaI site had been added and a HindIII site had been deleted at the 3′ end (5′-GCT CTA GAT CAT GCT TTA AAA CTT GTG GCC AC-3′: SEQ ID NO:18). This PCR product was subcloned into pCR2.1-TOPO (Invitrogen), and subsequently, a fragment was cut out at the EcoRI and XbaI sites and inserted at the EcoRI/XbaI sites of a yeast expression vector, pYES2/CT vector (Invitrogen) to make pYES2/CT-DL having a GAL1 promoter (a gene sequence of the expressed protein: SEQ ID NO:19 and an amino acid sequence thereof: SEQ ID NO:20). The yeast INVSc1 (Invitrogen) was transfected with this vector by an electroporation method, and cultured in an SD agar medium for 4 days. A pYES2/CT-DL-introduced strain was cultured in a BMD liquid medium for one day, then the expression was induced by adding 15% glycerol, and the cells were collected at 5 and 8 hours after the induction. The collected yeast cells were washed with sterile water, and disrupted with glass beads in 20 mM Tris buffer (pH 7.5) containing 1 mM EDTA, 50 mM KCl, 5% glycerol and an inhibitor cocktail, Complate Mini (Roche) to collect lysate. To 50 μL of the collected lysate, 50 μL of 14 μM dinoflagellate luciferin (1 M phosphate buffer (pH 6.0)) was added, and the luminescence activity for 20 sec was determined using AB2200 (ATTO) (FIG. 13). The dinoflagellate luciferase was expressed in the yeast after 5 hours, and had the luminescence activity. The present result indicates that the dinoflagellate luciferase is useful as the reporter enzyme in the yeast.

Also, pYES2/CT-SDL vector where pYES2/CT-DL yeast secretion signal having the GAL1 promoter (Clements M. J., Gatlin G. H., Price M. J., Edwards R. M.: Secretion of human epidermal growth factor Saccharomyces cerevisiae using synthetic leader sequences. Gene, 106:267-272, 1991) had been introduced was constructed (gene sequence: SEQ ID NO:21, amino acid sequence thereof: SEQ ID NO:22). If the yeast INVSc1 (Invitrogen) is transfected with this vector by the electroporation as with the above, it will be possible to construct a yeast expression system having a secretion property.

### INDUSTRIAL APPLICABILITY

In the particularly preferable embodiment in the present invention, the blue light-emitting enzyme derived from the dinoflagellate can be also expressed in the eukaryote, and the maximum amount of emitted light can be obtained under the appropriate condition. By the use of this system, the transcription activities in the eukaryotic cells can be simultaneously determined as the blue light-emitting enzymes in the multiple gene expression detection system. These can be utilized for the treatment/examination of pathology and the new drug development.

The gene construct of the invention can be used for the promoter assay system by being introduced into the eukaryotic cells such as yeast cells and mammalian cells including human cells.

Genetic characters of the yeast have been elucidated in detail, and many translational modifications have been known. The yeast can rapidly grow in the medium defined as the eukaryote of a single-cell organism, and can be more easily handled compared to the other eukaryotes. Thus recently, the yeast has been actively used for influence evaluation of endocrine disruptors on the cells and cytotoxicity evaluation/screening of drugs.

Therefore, according to the present invention, it is possible to perform the influence evaluation of endocrine disruptors on the cells and the cytotoxicity evaluation/screening of drugs by the use of not only the mammalian cells such as human cells precisely but also the yeast simply.

1. A gene construct, comprising a eukaryotic promoter operably linked to a gene selected from the group consisting of:
(a) a gene encoding an active light-emitting polypeptide, said polypeptide having a sequence of SEQ ID NO:2 or an active-light-emitting fragment thereof;
(b) a complement of (a), and
(c) a gene capable of hybridizing with (a) or (b) at 37° C. in 0.2×SSC with 0.1% SDS.

2. The gene construct according to claim 1 wherein the gene encodes a polypeptide fragment having a sequence selected from the group consisting of SEQ ID NO: 4, SEQ ID NO: 6, SEQ ID NO: 8 and SEQ ID NO: 10.

3. The gene construct according to claim claim 2, wherein the gene has a sequence selected from the group consisting of SEQ ID NO:3, SEQ ID NO:5, SEQ ID NO:7, and SEQ ID NO:9.

4. The gene construct according to claim 1, wherein the gene has a sequence of SEQ ID NO: 1.

5. The gene construct according to claim 1, wherein said construct incorporating the light-emitting enzyme to be capable of secreting.

6. An expression vector comprising the gene construct according to claim 1.

7. A eukaryotic cell transformed with the gene construct according to claim 1 or the expression vector according to claim 6.

8. A luminescence determining reagent comprising dinoflagellate luciferin and adjusted to pH 5 to 7.5 for determining an enzyme activity of an expressed light-emitting enzyme from luminescent dinoflagellate.

9. The luminescence determining reagent according to claim 8 having a buffer with pH 5 to 7.5.

10. The luminescence determining reagent according to claim 8 wherein a concentration of the dinoflagellate luciferin is adjusted to 10 to 30 μM.

11. A luminescence determining reagent wherein dinoflagellate luciferin and Firefly luciferin coexist in order to simultaneously determine one or more expressed light-emitting enzymes from luminescent dinoflagellate and one or more expressed luciferases from luminescent beetle.

12. A method for determining of a gene transcription activity of a promoter linked to a light-emitting enzyme gene, wherein the eukaryotic cells according to claim 7 are cultured, and a luminescence activity in the cultured eukaryotic cells or a disruption solution thereof is determined in the presence of the luminescence determining reagent comprising dinoflagellate luciferin and adjusted to pH 5 to 7.5 for determining an enzyme activity of an expressed light-emitting enzyme from luminescent dinoflagellate.

13. The method of claim 12, wherein said dinoflagellate luciferin is adjusted to 10 to 30 μM.

14. The method of claim 12, wherein said dinoflagellate luciferin coexists with Firefly luciferin in order to simultaneously determine one or more expressed light-emitting enzymes from luminescent dinoflagellate and one or more expressed luciferases from luminescent beetle.

15. The gene construct of claim 1, further comprising a second light-emitting enzyme genes incorporated under the control of the distinct promoter, wherein said second light emitting enzyme comprises one or more genes encoding luciferase derived from luminescent beetle.
